(19) United States
(12) Patent Application Publication
(10) Pub. No.: US 2024/0274260 A1
(43) Pub. Date: Aug. 15, 2024

(54) ACUPOINT POSITIONING SYSTEM AND ACUPOINT POSITIONING METHOD

(71) Applicant: Asia Eastern University of Science and Technology, NEW TAIPEI CITY (TW)

(72) Inventor: PENG-WEN CHEN, New Taipei City (TW)

(21) Appl. No.: 18/408,662

(22) Filed: Jan. 10, 2024

(30) Foreign Application Priority Data
Feb. 9, 2023 (TW) 112104506

(51) Int. Cl. G16H 20/30

(52) U.S. Cl. CPC G16H 20/30

(57) ABSTRACT

An acupoint positioning system and an acupoint positioning method are provided. The acupoint positioning system includes a server system and an electronic device. The server system stores predetermined human body model data. The electronic device captures a human body part image and transmits the human body part image to the server system. The server system calibrates the predetermined human body model data based on human body size data of the human body part image, and, based on a calibration result, generates human body model data that includes a human body massage part and at least one piece of human body massage positioning data located at the human body massage part. The server system transmits the human body model data to the electronic device that displays the human body model data.

### CROSS-REFERENCE TO RELATED PATENT APPLICATION

This application claims the benefit of priority to Taiwan Patent Application No. 112104506, filed on Feb. 9, 2023. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a generation of massage part data, and more particularly to an acupoint positioning system and an acupoint positioning method.

### BACKGROUND OF THE DISCLOSURE

Modern people often suffer from sinew, bone, or muscular discomfort due to sedentary lifestyles, where certain postures need to be maintained over a long time. Such discomfort caused by incorrect body postures or sports injuries are can be relieved through self-administered massages, or by having a massage therapist perform a massage.

However, even with the assistance of a massage devices (e.g., electric massagers or massage guns), only the force of the massage is strengthened, while exact parts of the body that need to be massaged remain difficult to be identified when using such machines.

### SUMMARY OF THE DISCLOSURE

In response to the above-referenced technical inadequacies, the present disclosure provides an acupoint positioning system and an acupoint positioning method to address the lack of massaging knowledge of the general population, and to quickly locate massage parts or positions for performing massage.

In order to solve the above-mentioned problems, one of the technical aspects adopted by the present disclosure is to provide an acupoint positioning method for being applied to a server system having a database that stores predetermined human body model data. The acupoint positioning method includes steps of: capturing, via an image capture member of an electronic device, a human body part image; transmitting, via the electronic device, the human body part image to the server system; calibrating, via the server system, the predetermined human body model data in the server system based on at least one piece of human body size data of the human body part image; generating, via the server system, human body model data based on a calibration result, in which the human body model data includes a human body massage part and at least one piece of human body massage positioning data located at the human body massage part; and transmitting, via the server system, the human body model data to the electronic device; and displaying, via the electronic device, the human body model data.

In order to solve the above-mentioned problems, another one of the technical aspects adopted by the present disclosure is to provide an acupoint positioning system. The acupoint positioning system includes a server system and an electronic device. The server system has a database that stores predetermined human body model data. The electronic device is communicatively connected to the server system. The electronic device includes an image capture member that captures a human body part image, and the electronic device transmits the human body part image to the server system. The server system calibrates the predetermined human body model data based on at least one piece of human body size data of the human body part image, and the server system generates human body model data based on a calibration result. The human body model data includes a human body massage part and at least one piece of human body massage positioning data located at the human body massage part, and the server system transmits the human body model data to the electronic device. The electronic device displays the human body model data.

Therefore, in the acupoint positioning method and the acupoint positioning system provided by the present disclosure, image data can be provided for generating data for a massage part, so as to achieve the effect of an interactive massage. Furthermore, by positioning massage positions of an entire human body via an augmented reality (AR) manner and using an AR visual guidance, a body part that requires massage can be more accurately located.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a flowchart of an acupoint positioning method according to one embodiment of the present disclosure;

FIG. 2 is a flowchart of a step for generating human body massage positioning data of FIG. 1;

FIG. 3 is a schematic view of predetermined human body model data according to one embodiment of the present disclosure;

FIG. 4 is a schematic block diagram of an acupoint positioning system according to one embodiment of the present disclosure;

FIG. 5 illustrates a server system calibrating the predetermined human body model data according to one embodiment of the present disclosure;

FIG. 6 illustrates the server system generating the human body massage positioning data to provide image data according to one embodiment of the present disclosure;

FIG. 7 illustrates the server system calibrating first predetermined massage positioning data and second predetermined massage positioning data according to one embodiment of the present disclosure;

FIG. 8 illustrates the server system positioning the human body massage positioning data according to one embodiment of the present disclosure;

FIG. 9 illustrates an electronic device displaying an integrated image through an augmented reality program according to one embodiment of the present disclosure; and

FIG. 10 illustrates a process of forming the human body massage positioning data via a proportional bone measurement manner according to one embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### Embodiments

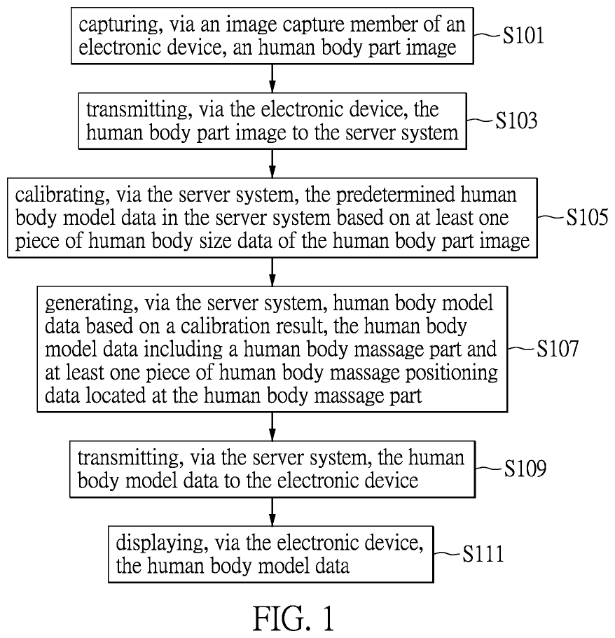

Referring to FIG. 1, an acupoint positioning method is applied to a server system. The server system has a database, and the database stores predetermined human body model data that is related to a massage for a human body. The data that is obtained and processed in the electronic device relates to real user data. The acupoint positioning method includes steps as follows: step S101: capturing, via an image capture member of an electronic device, a human body part image; step S103: transmitting, via the electronic device, the human body part image to the server system; step S105: calibrating, via the server system, the predetermined human body model data in the server system based on at least one piece of human body size data of the human body part image; step S107: generating, via the server system, human body model data based on a calibration result, the human body model data including a human body massage part (e.g., the calf) and at least one piece of human body massage positioning data (e.g., the plurality of acupoint positions) located at the human body massage part; step S109: transmitting, via the server system, the human body model data to the electronic device; and step S111: displaying, via the electronic device, the human body model data.

For example, the predetermined human body model data include first predetermined massage positioning data and second predetermined massage positioning data. The first predetermined massage positioning data or the second predetermined massage positioning data includes one or a combination of acupoint positioning data and meridian positioning data. The first predetermined massage positioning data and the second predetermined massage positioning data have a correlation therebetween. The correlation is such as a relation of multiple acupoints (such as two acupoints) that are adjacent to or affected by two meridians of the human body. The predetermined human body model data can include multiple predetermined massage positioning data other than the first predetermined massage positioning data and the second predetermined massage positioning data.

When the first predetermined massage positioning data and the second predetermined massage positioning data are both acupoint positioning data, or when one of the first predetermined massage positioning data and the second predetermined massage positioning data is acupoint positioning data and the other is meridian positioning data, and when the acupoint positioning data relates to the meridian positioning data similar to an acupoint relating to a meridian, the server system generates corresponding massage part data according to a positioning result. The predetermined massage positioning data can also include a group of acupoints that can be marked using numbers for indicating massage priority or categorized using colors.

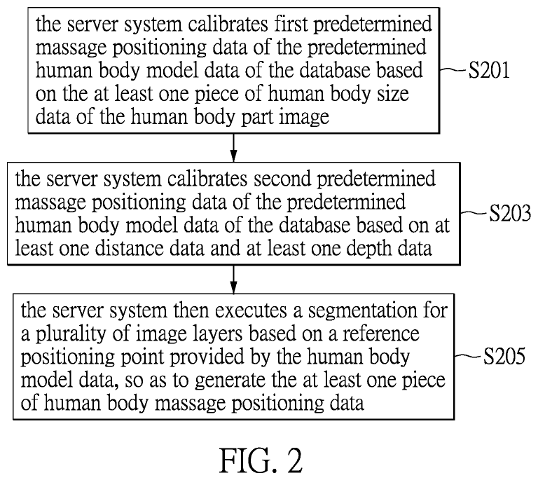

Referring to FIG. 2, the aforementioned step S105 further includes following steps: step S201: the server system calibrates first predetermined massage positioning data of the predetermined human body model data of the database based on the at least one piece of human body size data of the human body part image; step S203: the server system calibrates second predetermined massage positioning data of the predetermined human body model data of the database based on at least one distance data and at least one depth data; and step S205: the server system then executes a segmentation for a plurality of image layers based on a reference positioning point provided by the human body model data, so as to generate the at least one piece of human body massage positioning data.

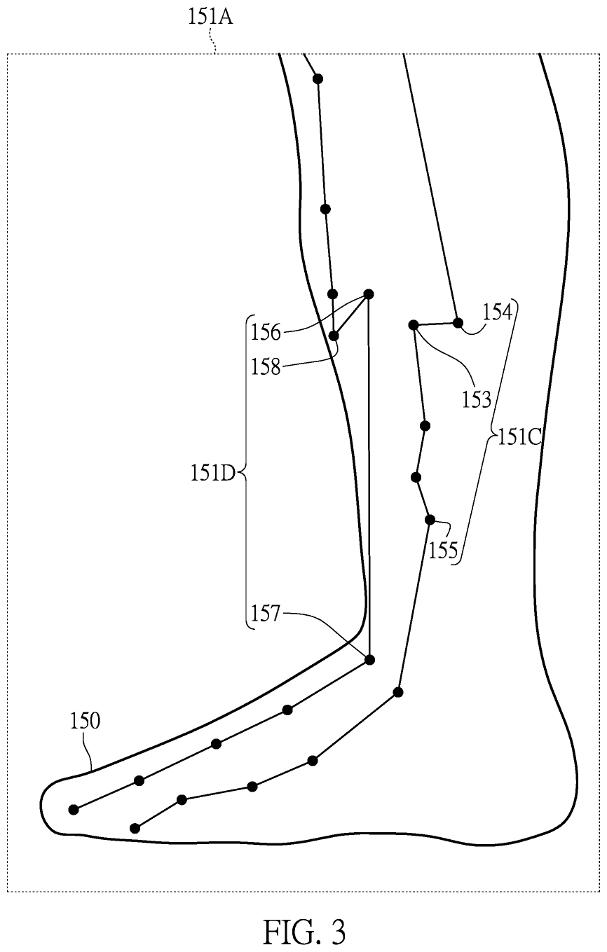

As shown in FIG. 3, a predetermined human body massage part 150 (e.g., a left calf) has first predetermined massage positioning data 151C and second predetermined massage positioning data 151D. The first predetermined massage positioning data 151C can include a positioning position of a first meridian that has a predetermined first acupoint 153, a predetermined third acupoint 154, and a predetermined fourth acupoint 155, the second predetermined massage positioning data 151D can include a positioning position of a second meridian that has a predetermined second acupoint 156, a predetermined fifth acupoint 157, and a predetermined sixth acupoint 158, and the first and second predetermined massage positioning data 151C and 151D can be illustrated by using curves or lines.

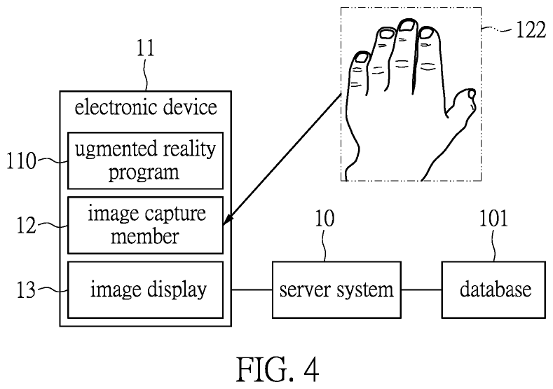

As shown in FIG. 4, an image capture member 12 of the electronic device 11 captures a human body part image 122 and inputs the human body part image 122 into a server system 10. The human body part image 122 can be an image of a hand, a calf, or other parts of a human body. In this embodiment, the human body part image 122 is an image of an open hand.

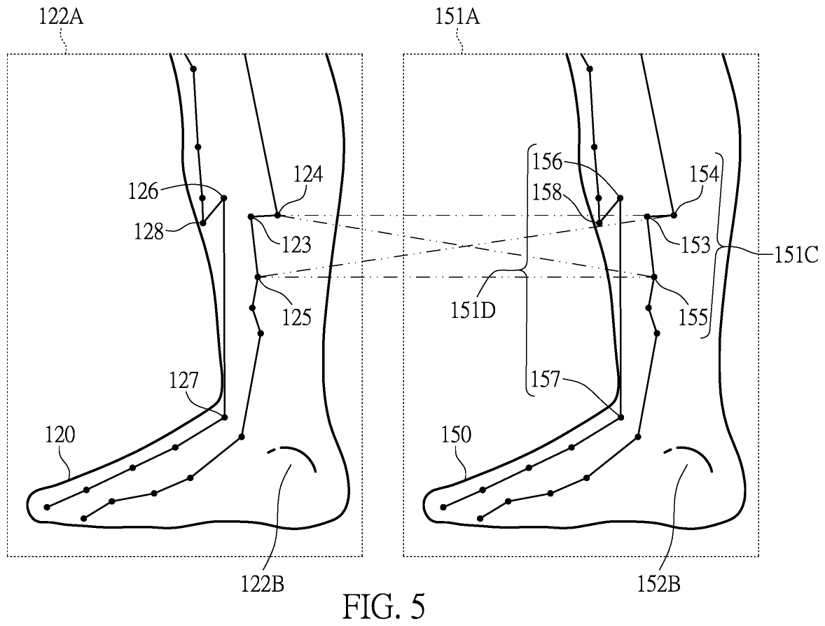

As shown in FIG. 5, the server system 10 can calculate human body size data 122A by positioning the acupoints using the proportional body measurement manner, also referred to as a proportional finger measurement manner. The human body size data 122A includes a human body massage part 120 (e.g., the left calf). Assuming that a width of the thumb in the human body part image 122 is defined as one inch wide or a width of the four fingers held together is defined as three inches wide, a position of a first acupoint 123 on the human body massage part 120 being at a Waiqiu acupoint can be deduced by one or a combination of the two defined lengths. That is to say, the position of the first acupoint 123 is deduced by the width of the thumb being defined as one inch wide or by the width of the four fingers held together being defined as three inches wide. In other words, the first acupoint 123 is the simulated position of the Waiqiu acupoint, and the first acupoint 123 such as the Waiqiu acupoint can be found at a position at the outer and lateral surface of the calf, seven inches above the tip of the ankle, and in the depression of the front edge of the fibula. Furthermore, a position of a second acupoint 126, also referred to as a Fenglong acupoint, on the human body massage part 120 can be found at a position that is moved from the first acupoint 123 leftward or upper-leftward by one inch or two inches by the width of the thumb being defined as one inch wide, or the position of a second acupoint 126, also referred to as the Fenglong acupoint, can be found at a position at the outer and lateral surface of the calf, eight inches above the tip of the ankle, and a width of two fingers outward from the front edge of the fibula by the width of the thumb being defined as one inch wide or by the width of the four fingers held together being defined as three inches wide.

By defining the acupoints of the two meridians corresponding to the first acupoint 123 and the second acupoint 126, and defining distances and positions between acupoints of each of the meridians, the position of a first meridian having the first acupoint 123, a third acupoint 124, and a fourth acupoint 125 on the human body massage part 120 and the position of a second meridian having the second acupoint 126, a fifth acupoint 127, and a sixth acupoint 128 on the human body massage part 120 can be located, and be expressed using curves or lines.

The width of the thumb or the width of the four fingers held together is different for each person. Therefore, for each person, when the image capture member 12 of the electronic device 11 captures the human body part image 122 and inputs the human body part image 122 into the server system 10 to locate the first acupoint 123 to the sixth acupoint 128, positioning data of the human body size data 122A is different. When the server system 10 obtains the positioning data of the human body size data 122A based on the human body part image 122, the first predetermined massage positioning data 151C and the second predetermined massage positioning data 151D of predetermined human body model data 151A can be calibrated according to a proportionality relationship.

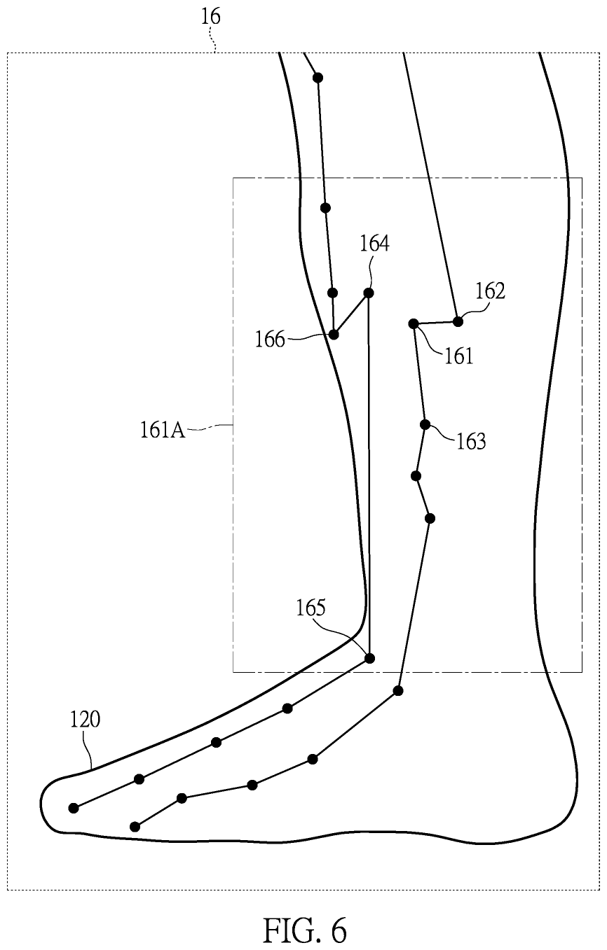

As shown in FIG. 6, based on the calibration results, the server system 10 generates and stores corresponding human body massage positioning data 161A of human body model data 16. The human body massage positioning data 161A is obtained by the server system 10 based on a relationship between the first predetermined massage positioning data 151C and the second predetermined massage positioning data 151D, where the relationship relates to distance data, orientation data, height data, or depth data.

Referring to FIG. 4 and FIG. 5, the human body massage part 120 of the human body size data 122A is assumed to be smaller or greater than the predetermined human body massage part 150 of the predetermined human body model data 151A. In this embodiment, only a case where the human body massage part 120 of the human body size data 122A is smaller than the predetermined human body massage part 150 of the predetermined human body model data 151A is illustrated.

When a height of an outer ankle tip position 122B of the human body massage part 120 in the human body size data 122A is three inches high, and a height of an outer ankle tip position 152B of the predetermined human body massage part 150 in the predetermined human body model data 151A is four inches high, a calibration of the predetermined human body model data 151A can be executed by proportional scaling using a three-quarter of the distance between position of the proportionality relationship. The position that is calibrated may be the outer ankle tip point, and the calibration process may be performed together with proportional scaling of the first predetermined massage positioning data 151C and the second predetermined massage positioning data 151D in the predetermined human body model data 151A to shorten (i.e., calibrate) a distance between the positions of the first predetermined massage positioning data 151C and the second predetermined massage positioning data 151D, so as to generate massage positioning data. The server system 10 can generate corresponding human body model data 16 (as shown in FIG. 6) based on the calibration results. The corresponding human body massage positioning data 161A in the human body model data 16 includes positioning information of the personalized human body massage part 120.

When the server system 10 calculates a positioning position of a first meridian having the first acupoint 123, the third acupoint 124, and the fourth acupoint 125 based on the human body size data 122A, a positioning position (i.e., positioning data of coordinates) of a second meridian having the second acupoint 126, the fifth acupoint 127, and the sixth acupoint 128 can be calculated based on a triangulation ratio (as indicated by the broken line in FIG. 5). Accordingly, the positioning position of the second meridian is then deduced and predicted, and includes a curved meridian path or a lined meridian path.

As shown in FIG. 4, when the electronic device 11 is communicatively connected to the server system 10 and the electronic device 11 includes an image capture member 12 and an image display 13, an interactive massage can be performed by using personalized massage part information. The image capture member 12 of the electronic device 11 captures a human body part image 122, and after the server system 10 receives the human body part image 122, the server system 10 provides the human body model data 16 to the electronic device 11. When the electronic device 11 receives the human body model data 16, the image display 13 of the electronic device 11 displays the human body model data 16. In this way, a user can perform the interactive massage by the human body model data 16 being displayed by the image display 13.

The electronic device 11 further includes an augmented reality program 110. The augmented reality program 110 of the electronic device 11 projects the human body model data 16 provided by the server system 10 into a spatial environment, and the user performs an interactive massage based on the human body model data 16 projected into the spatial environment. The human body massage positioning data 161A (FIG. 6) of the human body model data 16 includes a plurality of coordinate positioning data 161 to 166, and the coordinate positioning data 161 to 166 of the human body model data 16 is a group of acupoints on which a massage sequence can be marked. In addition, the group of acupoints can be displayed in various ways, for example, the group of acupoints can be completely displayed, or displayed by recommendation, or customized to be displayed as having different colors and importance indicators.

Referring to FIG. 5 and FIG. 6, the server system 10 calibrates the first predetermined massage positioning data 151C of the predetermined human body model data 151A based on the human body size data 122A of the human body part image 122, and then calibrates the second predetermined massage positioning data 151D of the predetermined human body model data 151A based on at least one distance data and/or at least one depth data. The server system 10 generates the human body massage positioning data 161A based on the calibration results, and the human body massage positioning data 161A is a three-dimensional positioning image with respect to the human body massage part.

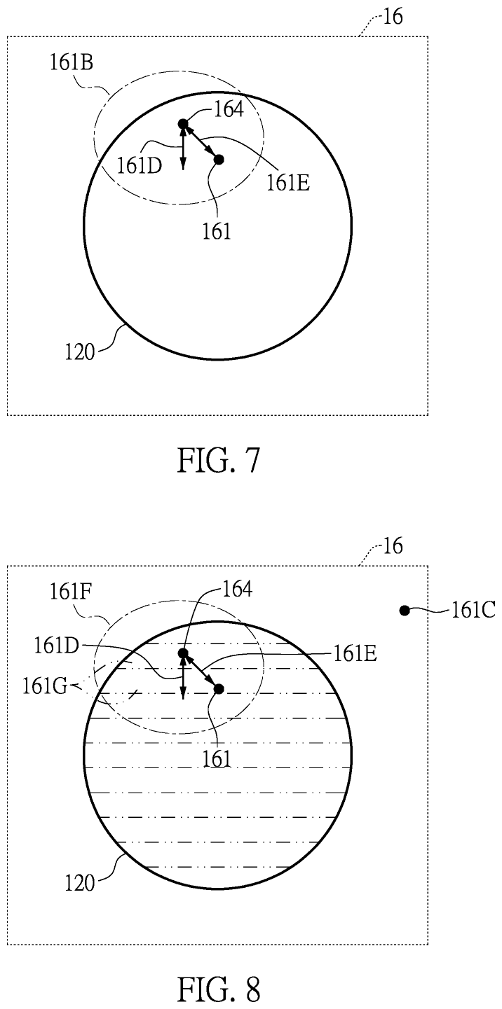

Reference is further made to FIG. 5, FIG. 6, and FIG. 7. When the server system 10 deduces that the position of the first acupoint 123 is the Waiqiu acupoint of the human body massage part 120 based on the human body size data 122A in the human body part image 122, first coordinate positioning data 161 of human body massage positioning data 161B is then generated based on the calibration. In addition, by using a reference point of the first acupoint 123 of the human body size data 122A, the server system 10 can calibrate the predetermined second acupoint 156 in the second predetermined massage positioning data 151D based on at least one distance data 161E and at least one depth data 161D.

As shown in FIG. 7, the server system 10 is able to obtain second coordinate positioning data 164 based on a height or the depth data 161D of the first coordinate positioning data 161, such that the server system 10 is able to provide the human body massage positioning data 161B of the human body model data 16, and the human body massage positioning data 161B is a three-dimensional positioning image relative to the human body massage part 120.

Referring to FIG. 8, the server system 10 calibrates the first predetermined massage positioning data 151C of the predetermined human body model data 151A based on the human body size data 122A of the human body part image 122, and the server system 10 then calibrates the second predetermined massage positioning data 151D based on the distance data and/or the depth data. The server system 10 then provides a reference positioning point 161C based on the human body model data 16 to generate corresponding three-dimensional image data. Afterwards, the server system 10 executes a segmentation for a plurality of image layers 161G based on the reference positioning point 161C to locate human body massage positioning data 161F. The depth data or the depth-related description may be the depth of inserting needles under the principle of acupuncture, and be used to locate positions of acupoints. The positions of the acupoints may be located in the depressions of the muscles or the bones and have a depth. Specifically, the segmentation of the plurality of image layers 161G refers to that when an acupoint is displayed on a human body model, since the human body has curved surfaces, the depth has to be taken into account when positioning the acupoints. For example, some of the acupoints that are displayed as being on a front side of the arm actually have depths that can be referred to on lateral sides of the arm, so the user needs to rotate the arm to the lateral sides for the depth of the acupoints to be seen.

The reference positioning point 161C is a spatial coordinate of an origin point in the human body model data 16. Therefore, the first coordinate positioning data 161 and the second coordinate positioning data 164 in the human body massage positioning data 161F are the relative positions of the reference positioning point 161C. The server system 10 may execute a segmentation calculation for the image layers 161G based on the reference positioning point 161C provided by the human body model data 16 to locate the specific positions of the first coordinate positioning data 161 and the second coordinate positioning data 164.

When the reference positioning point 161C is on a horizontal plane, the server system 10 may rotate or flip the image of the human body model data 16 according to the reference positioning point 161C for initializing image segmentation and executing analysis and calculation, in order to determine whether or not some of the image layers 161G include the first coordinate positioning data 161 or the second coordinate positioning data 164.

When each of the image layers 161G is segmented and located, each of the image layers 161G can be provided without the first coordinate positioning data 161 or the second coordinate positioning data 164. In other words, the server system 10 uses the reference positioning point 161C and the segmentation of the plurality of image layers 161G to generate multiple planes or individual distances between the plurality of image layers 161G, so as to locate (mark) the position of the first coordinate positioning data 161 or the second coordinate positioning data 164. Moreover, each of the image layers 161G can be spaced apart at a predetermined distance for image division on each of the image layers 161G.

Referring to FIG. 3 to FIG. 5, the server system 10 can standardize the massage part positioning information in the database 101 based on the predetermined human body model data 151A. The predetermined human body model data 151A includes the first and second predetermined massage positioning data 151C and 151D. The predetermined human body model data 151A can be utilized by the server system 10 that uses graphical description software for creating a coordinate model graphic or a three-dimensional model graphic, and the graphic can be three-dimensional image data. The predetermined human body model data 151A can also be constructed as a machine-learning model. In the present disclosure, the machine-learning model can calibrate predetermined acupoint data based on acupoint calibration data.

After the server system 10 builds a well-established database 101 for massage positioning, the server system 10 is able to capture the human body part image 122 through the image capture member 12 of the electronic device 11 to obtain at least one piece of human body size data 122A (as shown in FIG. 5). The electronic device 11 is, for example, a mobile phone, a tablet computer, or a desktop computer.

By having the image capture member 12 scanning the human body part and capturing the human body part image 122 and the electronic device 11 interacting with the user, the human body massage positioning data 161A on the body part, such as the human body massage part 120, can be displayed on the image display 13 of the electronic device 11 in real time. The process of displaying the human body massage positioning data 161A can be carried out as shown in FIG. 6, in which the first coordinate positioning data 161, third coordinate positioning data 162, fourth coordinate positioning data 163, a meridian corresponding to the first coordinate positioning data 161, the second coordinate positioning data 164, fifth coordinate positioning data 165, and sixth coordinate positioning data 166, and a meridian corresponding to the second coordinate positioning data 164 are presented through real-time image visualization. Accordingly, the user can be immediately informed of the position information such as the relationship of the relevant acupoints on each of the meridians of the human body massage positioning data 161A on the human body massage part 120, the relationship of the relevant meridians to each other, or the relationship of all of the relevant acupoints on the relevant meridians to each other, and so on. In the present disclosure, the acupoint relationships of the Waiqiu and Fenglong acupoints as represented by the first coordinate positioning data 161 and the second coordinate positioning data 164 are taken as an example. In other embodiments, the present disclosure may be based on the example of the relevant meridians of a gallbladder meridian and a stomach meridian respectively represented by the Waiqiu and Fenglong acupoints, as well as all the relevant acupoints in the respective meridians, and the present disclosure is not limited thereto. In other words, the user can interact with the system through a handheld device to minimize reference point errors and can further switch display planes.

The server system 10 can further match the relevance of one or more pieces of ailment composite data to the first and second predetermined massage positioning data 151C and 151D in the predetermined human body model data 151A. When a filtering module is provided in the server system 10 to look for information about a particular ailment composite data or massage part in the predetermined human body model data 151A, the server system 10 is able to obtain the human body massage positioning data 161A related with the corresponding ailment or the ailment to which the human body massage positioning data 161A corresponds based on the calibration results. The filtering module may also be a software program of the electronic device 11 or a module that executes a filtering program through configuration of certain conditions in a computer.

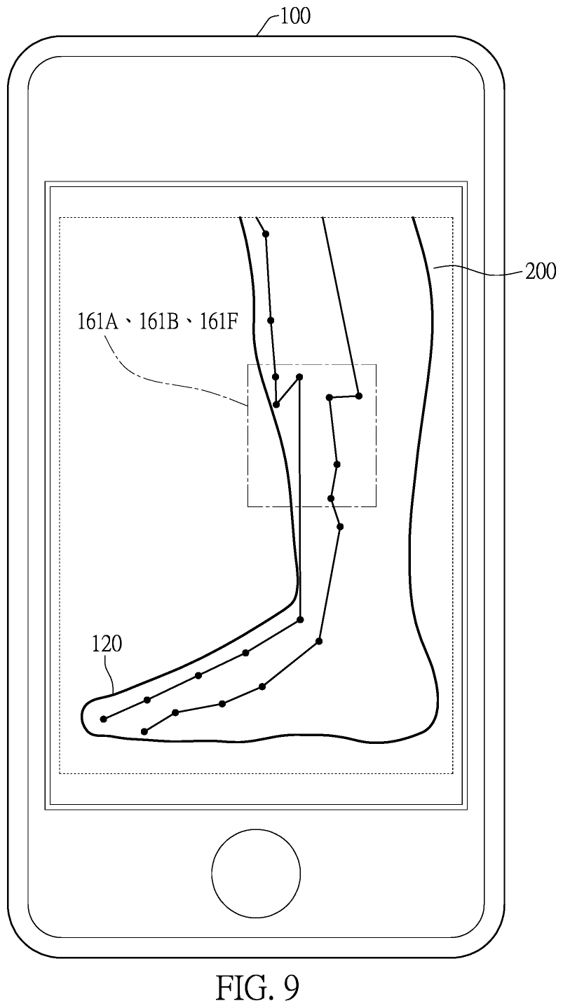

Reference is further made to FIG. 4 and FIG. 9. The electronic device 11, such as a mobile phone 100, can display an integrated image 200 through the augmented reality program 110, and the integrated image 200 includes the human body massage part 120 and a plurality of human body massage positioning data 161A, 161B, and 161F located on the human body massage part 120. The human body massage positioning data 161A, 161B, and 161F form geographically similar massage positioning data or massage positioning data that has correlation and influence.

When the present disclosure integrates virtual and actual images, information about a specific ailment and the corresponding massage positioning information can be displayed on the image display 13. The aforementioned filtering module (not shown in the figures) may be configured according to conditions to filter the database 101, where the conditions can be food therapeutic information corresponding to a specific ailment or a specific massage position, and the present disclosure is not limited thereto.

As shown in FIG. 8 and FIG. 9, when the lens of the mobile phone 100 (or the image capture member 12) is focused on a human body part, an image of the human body massage part 120 is displayed. Furthermore, the image of the human body massage part 120 that is displayed is not a flat surface, but is a curved three-dimensional image. After the server system 10 generates the human body model data 16 and provides the reference positioning point 161C on the three-dimensional spatial model of the human body model data 16, the server system 10 executes a segmentation calculation for the plurality of image layers 161G based on the reference positioning point 161C in order to locate the human body massage positioning data 161A, 161B, and 161F (as shown in FIG. 6 to FIG. 8).

The augmented reality program 110 of the electronic device 11 (e.g., the mobile phone 100) displays an image of the human body massage part 120 for a screen-guided manner to retrieve a correct position of the human body massage part 120. The user can move a position or a direction of the human body massage part 120 to allow the image capture member 12 to capture an image and allow the server system 10 to obtain the human body size data 122A and generate the human body model data 16. Accordingly, when the user moves the position or the direction of the human body massage part 120, the server system 10 can execute the segmentation calculation for the plurality of image layers 161G according to the reference positioning point 161C of the human body model data 16 to locate the human body massage positioning data 161A, 161B, and 161F (as shown in FIG. 9).

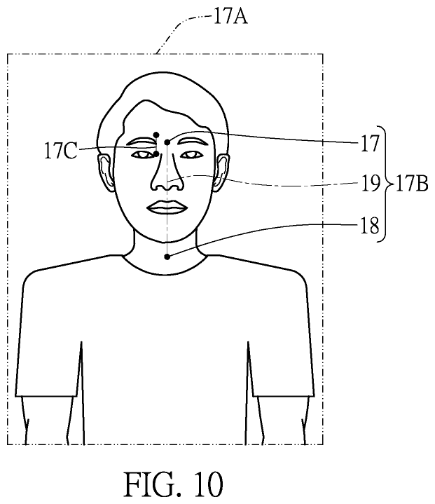

Referring to FIG. 4 and FIG. 10, images of human body parts at different positions are captured and input into the server system 10 to generate personalized virtual-real integrated massage part positioning data. For example, in order to form human body massage positioning data 17A about the head and upper half of the human body to include first massage positioning data 17, second massage positioning data 18, and meridian positioning data 19, images of the human body parts of the head and the upper half of the human body are captured by using the image capture member 12 and are input into the server system 10. Through the operation of the method or the system provided in the present disclosure, the first massage positioning data 17, the second massage positioning data 18, and the meridian positioning data 19 that are personalized can be generated.

Based on a manner of using the human body part image 122 to calculate the human body size data 122A, the server system 10 is assumed to use a proportional bone measurement manner to carry out the deduction (the proportional body measurement manner is used in the aforementioned description). That is, the server system 10 firstly locates a Yintang acupoint of the head (i.e., locates the first massage positioning data 17 that is, by definition, located one inch away from the two eyebrows). At the same time, by using the proportional bone measurement manner, a measurement is taken from the center between the eyebrows downwards by eighteen inches to the upper half of the human body to locate a Dazhui acupoint, such that the second massage positioning data 18 and the corresponding meridian positioning data 19 are located. The server system 10 can obtain the human body size data of the Yintang acupoint and the Dazhui acupoint and the meridian data between the Yintang acupoint and the Dazhui acupoint based on the proportional bone measurement manner, and after calibrating the corresponding acupoints in the predetermined human body model data, the server system 10 generates the corresponding human body massage positioning data 17A according to the calibration result. Therefore, the server system 10 can synthesize human body part images and the human body massage positioning data 17A of the head and the upper half of the human body, and the server system 10 then generates an integrated image data. The integrated image data is conceptually identical with integrated image data 200 of the aforementioned left calf (i.e., the human body massage part 120 as shown in FIG. 9) with the only difference being for which body part the integrated image data is to be formed.

In the present disclosure, after first massage positioning data 17B including the first massage positioning data 17, the second massage positioning data 18, and the meridian positioning data 19 is obtained, the corresponding adjacent or related acupoints of another meridian, and second massage positioning data 17C of each acupoint can also be obtained. The relationship between the first massage positioning data 17B and the second massage positioning data 17C may also be a relationship between any two of a Ren meridian, a Du meridian, a bladder meridian, and the gallbladder meridian of the head.

### Beneficial Effects of the Embodiments

One of the possible technical effects of the present disclosure is that, image data can be provided for generating the massage part data, so as to achieve the effect of an interactive massage. A predetermined or personalized whole body massage part positioning can be achieved through the augmented reality (AR) technology, and the user can more accurately find the massage part with the guidance of the augmented reality screen, where the massage part is such as a set of weighted and sequential acupoint group, but the present disclosure is not limited to.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. An acupoint positioning method for being applied to a server system having a database that stores predetermined human body model data, the acupoint positioning method comprising:
capturing, via an image capture member of an electronic device, a human body part image;
transmitting, via the electronic device, the human body part image to the server system;
calibrating, via the server system, the predetermined human body model data in the server system based on at least one piece of human body size data of the human body part image;
generating, via the server system, human body model data based on a calibration result, wherein the human body model data includes a human body massage part and at least one piece of human body massage positioning data located at the human body massage part; and transmitting, via the server system, the human body model data to the electronic device; and
displaying, via the electronic device, the human body model data.

2. The acupoint positioning method according to claim 1, wherein the process of displaying the human body model data via the electronic device includes displaying the human body model data by using an augmented reality program of the electronic device.

3. The acupoint positioning method according to claim 1, wherein the server system calibrates first predetermined massage positioning data of the predetermined human body model data based on the at least one piece of human body size data of the human body part image, and the server system then calibrates second predetermined massage positioning data based on at least one distance data and at least one depth data.

4. The acupoint positioning method according to claim 3, wherein, after the server system calibrates the second predetermined massage positioning data based on the at least one distance data and the at least one depth data, the server system then executes a segmentation for a plurality of image layers based on a reference positioning point provided by the human body model data, so as to generate the at least one piece of human body massage positioning data.

5. The acupoint positioning method according to claim 1, wherein the human body massage positioning data includes a plurality of coordinate positioning data, the coordinate positioning data is a group of acupoints, and a massage sequence is marked on the group of acupoints.

6. An acupoint positioning system, comprising:
a server system having a database that stores predetermined human body model data; and
an electronic device communicatively connected to the server system, wherein the electronic device includes an image capture member that captures a human body part image, and the electronic device transmits the human body part image to the server system;
wherein the server system calibrates the predetermined human body model data based on at least one piece of human body size data of the human body part image, and the server system generates human body model data based on a calibration result; wherein the human body model data includes a human body massage part and at least one piece of human body massage positioning data located at the human body massage part, and the server system transmits the human body model data to the electronic device; wherein the electronic device displays the human body model data.

7. The acupoint positioning system according to claim 6, wherein the server system calibrates first predetermined massage positioning data and second predetermined massage positioning data of the predetermined human body model data based on the at least one piece of human body size data of the human body part image.

8. The acupoint positioning system according to claim 7, wherein the server system calibrates the second predetermined massage positioning data based on distance data and/or depth data to generate the human body model data, and the server system executes a segmentation for a plurality of image layers based on a reference positioning point provided by the human body model data, so as to generate the at least one piece of human body massage positioning data.

9. The acupoint positioning system according to claim 8, wherein the electronic device includes an augmented reality program, and after the electronic device receives the human body model data from the server system, the electronic device displays the human body model data via the augmented reality program.

10. The acupoint positioning system according to claim 6, wherein the predetermined human body model data is a machine-learning model.
